SEQUENCE LISTING (14 sequences)

SEQ ID NO: 1 - DNA, 42 bp, Artificial Sequence
  Primer
tattatgagctcgagatacgttctccagcgtatgtatttcat

SEQ ID NO: 2 - DNA, 49 bp, Artificial Sequence
  Primer
tattatactagttctcgtctttgttgatgttttatatcacaagatgtag

SEQ ID NO: 3 - DNA, 42 bp, Artificial Sequence
  Primer
tattatcccggggagattccaaactggttcattgaaataggc

SEQ ID NO: 4 - DNA, 41 bp, Artificial Sequence
  Primer
tattatggatcccttattttatcctgcctgggttgagtgat

SEQ ID NO: 5 - DNA, 36 bp, Artificial Sequence
  Primer
tattatgagctcggtgctgtgaccgtttccaatacg

SEQ ID NO: 6 - DNA, 44 bp, Artificial Sequence
  Primer
tattatactagttggtatttctgatgatgttcttgctctctttg

SEQ ID NO: 7 - DNA, 43 bp, Artificial Sequence
  Primer
tattatcccgggactgcaactattcttacaatctttcatttac

SEQ ID NO: 8 - DNA, 45 bp, Artificial Sequence
  Primer
tattatggatcctttttacctaattactatgttttgaaacgttag

SEQ ID NO: 9 - DNA, 36 bp, Artificial Sequence
  Primer
tattatgagctctgttcacgccctctacgaaccatg

SEQ ID NO: 10 - DNA, 46 bp, Artificial Sequence
  Primer
tattatactagttagcgaggattgctgaaatcttgtatattttcag

SEQ ID NO: 11 - DNA, 26 bp, Artificial Sequence
  Primer
cccggggccacgttctaaactaacta

SEQ ID NO: 12 - DNA, 31 bp, Artificial Sequence
  Primer
atggatccttttttcttgtacttgtcttctc

SEQ ID NO: 13 - DNA, 537 bp, Saccharomyces cerevisiae
  CDS (1)..(537)
gcatccaacttgaacatttcgagaaagcttaccatatcaaccccatcatgctctttcgaaaattcaaatagcacatccattccttcgcccgcttcctcatctcaaagccacactccaatgagaaacatgagctcactctctgataacagcgttttcagccggaatatggaacaatcatcaccaatcactccaagtatgtaccaatttggtcagcagcagtcaaacagtatatgtggtagcaccgttagtgtgaatagtctggtgaatacaaataacaaacaaaggatctacgaacaaatcacgggtcctaacagcaataacgcaaccaatgattatattgatttgctaaacctaaatgagtctaacaaggaaaaccaaaatcccgcaacggcgcattacctcaatgggggcccacccaagacaagcttcattaaccatggaatgttcccctcgccaactgggaccataaatagcggtaaatctagcagtgcctcatctttaatttcttttggtatgggcaatacccaagtaatatag

SEQ ID NO: 14 - protein, 178 aa, Saccharomyces cerevisiae
ASNLNISRKLTISTPSCSFENSNSTSIPSPASSSQSHTPMRNMSSLSDNSVFSRNMEQSSPITPSMYQFGQQQSNSICGSTVSVNSLVNTNNKQRIYEQITGPNSNNATNDYIDLLNLNESNKENQNPATAHYLNGGPPKTSFINHGMFPSPTGTINSGKSSSASSLISFGMGNTQVI

(19) United States
(12) Patent Application Publication
(10) Pub. No.: US 2010/0221817 A1
(43) Pub. Date: Sep. 2, 2010

(54) Whole-Cell Sensor

(75) Inventors: Kai Ostermann, Dresden (DE); Wolfgang Pompe, Hartha (DE); Horst Böttcher, Dresden (DE); Gerhard Rödel, Karlsfeld (DE); Annett Gross, Dresden (DE)

Correspondence Address: GUDRUN E. HUCKETT DRAUDT, SCHUBERTSTR. 15A, WUPPERTAL, 42289, DE

(73) Assignee: TECHNISCHE UNIVERSITAET DRESDEN, Dresden (DE)

(21) Appl. No.: 12/597,296

(22) PCT Filed: Apr. 25, 2008
(86) PCT No.: PCT/EP2008/055099

§ 371 Date: May 7, 2010

(30) Foreign Application Priority Data
Apr. 25, 2007 (DE) 10 2007 020 725.7

(51) Int. Cl. C12M 1/34

(52) U.S. Cl. 435/288.7; 435/287.1

(57) ABSTRACT

The invention relates to whole-cell sensors for monitoring bioavailable nitrogen, phosphorus and sulphur, individually or in at least one combination in a medium, and to the use thereof. The whole-cell sensors consist of genetically modified yeast cells which are immobilised in a xerogel matrix and contain at least one marker gene controlled by a promoter of a gene, the transcription of said gene being significantly increased or reduced in the absence of nitrogen, phosphorus or sulphur, and the yeast cells are at least coupled to a signal detector.

The invention concerns whole-cell sensors for monitoring bio-available nitrogen, phosphorus, and/or sulfur in a medium and their use.

Hitherto existing solutions for monitoring bio-available nitrogen, phosphorus and/or sulfur in an aqueous medium are based on
the detection of the change of the physical or chemical properties of the medium with a change of the concentration of the analytes;
selective binding studies of the corresponding ions on suitable membranes or cage structures;
as well as specific reactions of the analytes resulting in the reaction products being detectable.

Based on this there are very differently designed solutions for electrical, electrochemical, optical or colorimetric sensors.

Known proposals for a solution have in common that none of them are capable of directly evaluating the immediate effect of nitrogen, phosphorus and/or sulfur compounds on processes in living cells, i.e., their biological availability. To the contrary, it is only possible to indirectly infer possible reactions of the microorganisms living in the medium based on the measured physical or chemical changes of the medium.

Boer et al. have examined the entire yeast genome with respect to yeast genes that are activated or repressed in response to the deficiency in bio-available nitrogen, phosphorus and/or sulfur. (Boer, V. M. et al., The genome-wider transcriptional responses of Saccharomyces cerevisiae grown on glucose in aerobic chemostat cultures limited for carbon, nitrogen, phosphorus, or sulfur, J. Biol. Chem. (2003) 278 (5) 3265-74). The expression profiles serve as indicator for characterization of nutrient-limited growth conditions.

EP 1426439 A1 discloses a method for detection of toxic substances. For this purpose, genetically modified yeast cells are used wherein the yeast cells contain a marker gene under the control of a promoter of a gene whose transcription is increased strongly in the presence of the toxic substance.

Inama et al. disclose methods for embedding living yeast cells in SiO₂ sol gel layers on glass supports, for example, for use as biocatalysts (Entrapment of viable microorganisms by SiO2 sol-gel layers on glass surfaces: trapping, catalytic performance and immobilization durability of Saccharomyces cerevisiae. J. Biotechnol. (1993) 30 (2) 197-210).

The invention disclosed in claim 1 has the object to develop a biosensor with which in a simple way a limitation of bio-available nitrogen, bio-available phosphorus and/or bio-available sulfur in a medium can be detected.

This object is solved with the features disclosed in claim 1.

The whole-cell sensor according to claim 1 is suitable for detection of bio-available nitrogen, phosphorus, and sulfur each individually or in at least one combination in a medium. It is comprised of gene-technologically modified yeast cells that are immobilized in a xerogel matrix, wherein the yeast cells contain at least one marker gene under the control of a promoter of a gene whose transcription is greatly increased or decreased in case of deficiency of nitrogen, phosphorus, or sulfur and wherein the yeast cells are coupled with the at least one signal detector.

The whole-cell sensors for bio-available nitrogen, phosphorus, and sulfur each individually or in at least one combination in a medium are characterized in particular in that a limitation of bio-available nitrogen, bio-available phosphorus and/or bio-available sulfur in the medium can be detected in a simple way.

The media in which the detection is realized are preferably aqueous media. They include, for example, culturing media that are used for culturing microorganisms such as bacteria or yeasts or algae or animal and plant cells. A wide array of applications for the biosensor are methods of white biotechnology where microorganisms are employed for producing special products such as special chemical compounds by means of biotransformation or biocatalysis. The biosensors may also be employed in fermentation processes in which microorganisms are used for producing foodstuff, for example, for brewing beer. Alternative fields of application are the purification of drinking water, industrial process water or wastewater in which the contents of bio-available nitrogen, phosphorus or sulfur is to be determined.

Living cells require that their demand of nitrogen, sulfur and phosphorus be covered by taking up corresponding nitrogen-, sulfur-, and phosphorus-containing compounds. The availability of such compounds in the medium is referred to in the following as bio-available nitrogen, sulfur or phosphorus. In contrast to measuring sensors that directly determine the nitrogen, phosphorus and/or sulfur contents of the medium to be examined by physical or physicochemical properties of the medium, the yeast cells according to the invention advantageously enable evaluation of the direct effect of nitrogen, phosphorus and/or sulfur compounds on processes in living cells, i.e., their biological availability. As yeast cells preferably Saccharomyces cerevisiae and Schizosaccharomyces pombe are used.

In this connection, at least one marker gene of the yeast cells is under the control of a promoter of a gene whose transcription greatly increases or greatly decreases in case of deficiency in nitrogen, phosphorus, or sulfur. The term greatly increased or greatly decreased transcription in this connection is to be understood as an at least two-fold increase or reduction of the transcription rate relative to transcription of the same promoter when sufficient bio-available nitrogen, phosphorus and/or sulfur is present. Marker genes in the context of the invention are genes that code for gene products whose activity leads to a physically measurable change. This physically measurable change depends on the change of the transcription rate. This can be detected by a suitable detection system in a simple and quick way. Preferred are such marker genes whose gene products can be detected without impairing the integrity or vitality of the cells.

The gene products are preferably proteins. Preferred are marker genes whose gene product is an enzyme that in the presence of a substrate catalyzes a color reaction. Further preferred are marker genes that code for a luciferase which in the presence of a substrate will emit light. Especially preferred are marker genes whose gene product is a protein that will fluoresce by excitation with light of a certain wavelength.

A promoter in genetics is a DNA sequence that regulates the expression of a gene. Promoters in the context of the present invention are preferably those regions of the genomic DNA that are responsible specifically for the regulation of the expression of a gene in that they react to specific intracellular or extracellular signals and, depending on the signals, activate or repress the expression of the gene that is under their control. In the method according to the invention these signals are the deficiency of bio-available nitrogen, sulfur and/or phosphorus. These regulating DNA regions in yeasts are in general located at the 5′ end of the start codon of the corresponding gene and have an average length of 309 by (Mewes H. W. et al., Overview of the yeast genome. Nature (1997) 387, 7-65). Such regulating regions can also be removed by more than 1,000 by from the coding sequence or can be located at the 3′ end of the coding sequence of the corresponding gene or even within the transcribing sequence of the corresponding gene. When such promoters are placed at the 5′ end of the start codon of any gene, preferably a marker gene, they regulate the activity of this gene as a function of the aforementioned specific signals.

The marker gene that is under the control of a promoter of a gene whose transcription is greatly increased to greatly reduced in case of deficiency in nitrogen, phosphorus or sulfur, is introduced into a yeast cell. In the yeast cell, it may be present on an extrachromosomal DNA molecule. Preferred in this context is a yeast expression vector that upon division of the yeast cell will replicate stably. Especially preferred is a so-called “high copy number” vector that is present in the yeast cell in a large number of copies. Alternatively, yeast artificial chromosomes may also be used as extrachromosomal DNA molecules.

In another embodiment, the marker gene together with the promoter is integrated into the chromosomal DNA of the yeast cell. In this way it is advantageously ensured that all progeny of the yeast cell also contain the marker gene under control of the specific promoter.

In order to detect the physically measurable change that is effected by the activity of the marker gene, the gene technologically modified cells are coupled with at least one signal detector. The type of signal detector is determined by the type of physically measurable change. As a signal detector preferably a photodetector or a spectrometer is used.

The sensor according to the invention is extremely robust. For example, when in a cell the expression of two proteins with different fluorescence whose fluorescence can be separated well with respect to measuring technology, is subjected to the control of a promoter that specifically greatly increases the transcription in case of a limitation and, on the other hand, in the same cell the expression of the second protein is subjected to the control of a promoter that greatly decreases the transcription, the possibility of a measuring error is greatly reduced.

In the whole-cell sensor according to the invention the yeast cells are immobilized in xerogels. Xerogels are gels that have lost their liquid for example by evaporation or suction. The gels are shape-stable, easily deformable disperse systems of at least two components that usually are comprised of a solid substance with long or strongly branched particles (for example, silicic acid, gelatin, collagens, polysaccharides, pectins, special polymers such as, polyacrylates, and other gelling agents that are often referred to as thickening agents) and a liquid (usually water) as a dispersion medium. In this connection, the solid substance in the dispersion medium provides a spatial network. When generating xerogel, the spatial arrangement of the net will change.

The use according to the invention of inorganic or biologically inert organic xerogels for embedding the yeast cells enables advantageously the survival of the cells while simultaneously the stability of the generated structures is ensured because they are toxicologically and biologically inert and generally are not decomposed by the yeasts. They enable furthermore advantageously embedding of nutrients and moisturizing agents that ensure survival of the cells.

Advantageous embodiments of the invention are provided in claims 2 to 22.

The yeast cells according to the invention are immobilized in a porous and optically transparent inorganic or biologically inert xerogel. According to the embodiment of claim 2 the xerogel is an inorganic xerogel of silicon dioxide, alkylated silicon dioxide, titanium dioxide, aluminum oxide or their mixtures; according to the embodiment of claim 3 the inorganic xerogel is produced preferably by a sol-gel process.

For this purpose, first silica or other inorganic nanosols are produced either by acid-catalyzed or alkali-catalyzed hydrolysis of the corresponding silicon oxide or metal oxide in water or a water-soluble organic solvent (such as ethanol). Preferably, the hydrolysis is carried out in water in order to prevent toxic effects of the solvent on the cells to be embedded. When producing nanosols by alkoxide hydrolysis, in the course of the reaction alcohols are produced that are subsequently evaporated from the obtained nanosol by passing through an inert gas stream and are replaced by water.

By the use of mixtures of different alkoxides the matrix properties can be affected in a targeted fashion. The sol-gel matrix enables advantageously the chemical modification by co-hydrolysis and co-condensation by using various metal oxides of metals such as Al, Ti, Zr for producing mixed oxides or of alkoxy silanes with organic groups at the Si atom for producing organically modified silicon oxide gels.

The cells to be embedded are mixed with the produced nanosol. The process of gel formation is preferably started by increasing the temperature, neutralizing the pH value, concentrating or adding catalysts such as fluorides. In this connection, the temperature should however not be increased to temperatures >42° C. in order not to damage the cells to be embedded. Upon transforming into a gel the nanosols reduce their surface area/volume ratio by aggregation and three-dimensional cross-linking. During this transformation of the nanosol into a so-called lyogel the cells are immobilized in the resulting inorganic network. The immobilization of survivable cells is advantageously controlled by the ratio of yeast cells/oxide and by addition of pore-forming agents.

The proportion of yeast cells based on the total quantity of the produced xerogel including the embedded cells, depending on the application, can be from 0.1 to 50% by weight. Preferred is a proportion of 2 to 25% by weight.

By drying, the solvent still contained in the lyogel is removed. In this way, the lyogel transforms into the xerogel. The resulting xerogel has a high porosity that enables a fast material exchange with the surrounding medium. The drying process causes great shrinkage of the gel that causes stress in the embedded cells. It is preferred that the drying step is therefore carried out in a gentle and slow way at temperatures of less than 40° C.

As the water contents of the matrix drops, the physiological activity and the survival rate of the embedded cells are reduced. However, a water contents that is too high however leads to low mechanical stability and reduces the durability of the structure.

The use of yeast cells in accordance with the present invention is therefore particularly advantageous because yeast cells have a high resistance with regard to dry conditions and even at minimal water contents do not lose their survivability. In this way it is possible to produce very dry xerogels.

The invention comprises also the use of different additives such as soluble organic salts, i.e., metal salts of organic carboxylic or sulfonic acids or open-chain or cyclic ammonium salts and quarternary salts of N-heterocyclic compounds as well as low-molecular polyanions or polycations or water-soluble organic compounds such as polycarboxylic acids, urea derivatives, carbohydrates, polyols, such as glycerin, polyethylene glycol and polyvinyl alcohol, or gelatin, that act as plasticizers, moisturizing agents and pore-forming agents, inhibit cell lysis, and considerably extend the survivability of the embedded cells.

The xerogel with the yeast cells according to the embodiment of claim 4 is applied to a substrate. In combination with the signal detector, preferably a photodetector, a functional element is provided in this way wherein the fluorescent light that is generated as a function of the bio-available analytes is converted by means of the photodetector into an electrical signal. Further, according to the embodiment of claim 5 the substrate is advantageously a light-guiding fiber, glass beads, a planar glass support or other shaped bodies of glass such as hollow spheres, rods, tubes or ceramic granules.

In this connection, the yeast cells are fixed in a porous and optically transparent xerogel, for example, a silicon dioxide xerogel. The silicon dioxide xerogel containing the microorganisms is deposited as a layer on glass beads, a light-guiding fiber, planar glass supports, or other shaped bodies such as hollow spheres, rods, tubes or ceramic granules by means of a known sol-gel process in that the nanosol-cell mixture is applied to the substrate to be coated or the substrate is immersed in the nanosol cell mixture and the nanosol is subsequently transformed by drying and the thus resulting concentration of the nanosol into the xerogel. The thus provided mechanical stability of these structures enables the introduction of the whole-cell sensor into a measuring system that can be immediately connected in the context of near-line diagnostics to the reaction space (fermenter) to be examined.

According to the embodiment of claim 6, the yeast cells are a component of an envelope structure that surrounds at least partially a cavity. This means that individual or several yeast cells are encapsulated in this cavity that has a porous envelope. The microporosity enables advantageously material exchange with the environment. In a further embodiment the envelope structure, according to the embodiment of claim 7, advantageously is embodied of a base body with an inner layer of a biological hydrogel and an outer layer of a porous and optically transparent xerogel wherein the layers are applied at least over portions thereof.

In this connection the yeast cells are embedded in the envelope structure (duplex embedding). The inner envelope is comprised of a biological hydrogel, for example alginate, and the exterior envelope is a porous xerogel layer, preferably an inorganic xerogel layer, especially preferred a silicon dioxide xerogel layer. The biological hydrogel stabilizes advantageously the yeast cells during the subsequent process of coating with the silicon dioxide sol and increases thus the survival probability of the cells. This duplex embedding can be advantageously realized by means of sequential coating by utilizing a nanoplotter. The mechanical stability of such structures enables the introduction of the whole-cell sensor into the reaction space (fermenter) to be examined in the context of near-line diagnostics.

The yeast cells are located according to the embodiment of claim 8 on at least one surface of a measuring cell that is transparent. The latter comprises moreover devices for supplying and removing the medium. According to the embodiment of claim 9 the measuring cell may be coupled to a heating device.

In this connection, the yeast cells are arranged in a temperature-controlled and light-microscopically observed measuring cell of a microfluidic system. The introduction of the medium into the measuring cell is realized by means of a microfluidic system (off-line diagnostics). The temperature adjustment is independent of the temperature adjustment in the fermenter.

According to the embodiment of claim 10, the signal detector is a photodetector. The photodetector is a solid-state image sensor with photoresistors, photodiodes or phototransistors connected to a data processing system. A solid-state image sensor is a flat and matrix-shaped arrangement of opto-electronic semiconductor elements acting as photoelectric receivers. The color and its intensity of the yeast cells are convertible into the equivalent electrical signals so that processing in the data processing system can be done.

In the beam path between the yeast cells and the photodetector there is at least one lens in accordance with the embodiment of claim 11. In this way, the light beams of the yeast cells can be focused on the photodetector so that a safe evaluation even of faint light changes is enabled.

The yeast cells according to the embodiment of claim 12 are coupled to an optical radiation source such that the radiation can impinge on the yeast cells and the yeast cells will fluoresce. The radiation source preferably provides electromagnetic rays as light in the visible range and the adjoining wavelength ranges in the infrared or ultraviolet spectrum. Preferably, this is an electromagnetic radiation source that emits light at a defined wavelength. The wavelength of the radiation source is selected based on the excitation spectrum of the fluorescent proteins.

The marker gene according to the embodiment of claim 13 is under the control of a promoter that is selected from the promoter of the genes YIR028W, YJR152W, YKR034W, YAR071W, YHR136C, YFL055W, YLL057C, NSR1, FET3, HIP1, YDR508C, RPS22B, YBRO99C, IPT1, SSU1, SOL1 and CTR1 of Saccharomyces cerevisiae.

Promoters in the context of the invention are also DNA regions that have in comparison to the corresponding yeast promoters a homology of more than 50%, preferably more than 80%. These regions can be derived, for example, from homologue genomic regions of other organisms, preferably other yeast strains. However, they may also be synthetically produced DNA sequences whose sequence exhibits homology of more than 50%, preferably more than 80%, match with the corresponding Saccharomyces cerevisiae promoter. Promoters can also be synthetic DNA sequences that are composed of a partial region of one of the aforementioned yeast promoters as well as a known basal promoter of Saccharomyces cerevisiae. The basal promoter provides the required DNA sequences for binding the transcription machinery while the partial sequences of the yeast promoter react specifically to regulating signals. Such a basal promoter is preferably the basal promoter of the cytochrome C-gene of Saccharomyces cerevisiae that comprises 300 by at the 5′ end of the start codon of cytochrome C-gene (Chen, J., et al. Binding of TFIID to the yeast CYC1 TATA boxes in yeast occurs independently of upstream activating sequences. PNAS (1994) 91:11909-11913).

Promoters of synthetic DNA sequences may contain also several regions of an identical DNA sequence. This multiplication of a regulatory DNA region enables advantageously an increase of the sensitivity of the promoter relative to the signals to be detected.

Promoters of genes whose transcription is greatly increased as a response to a corresponding limitation are advantageously in case of
nitrogen limitation: YIR028W, YJR152W and YKR034W
phosphorus limitation: YAR071W and YHR136C and
sulfur limitation YFL055W and YLL057C.

Promoters of genes whose transcription is greatly reduced as a response to a corresponding limitation are advantageously in case of
nitrogen limitation: NSR1, FET3, HIP1 and YDR508C
phosphorus limitation: RPS22B, YBRO99C and IPT1
sulfur limitation: SSU1, SOL1 and CTR1.

Preferred as a promoter is a DNA region that comprises up to 1,000 by at the 5′ end of the start codon of the gene controlled by it or a partial region of this DNA region that, upon limitation of nitrogen, phosphorus or sulfur, is capable of activating or suppressing the marker gene that is under the control of this sequence.

Especially preferred as promoters are those genomic regions that are enclosed by the primer pairs listed in Table 1. The underlined regions of the sequences are complementary to regions of the DNA to be amplified; the regions of the primers that are not underlined contain restriction sites for restriction enzymes for subsequent subcloning of the obtained PCR fragments.

TABLE 1

| | | | |
|---|---|---|---|
| | Seq. | | Seq. |
| Forward Primer Sequence | No. | Reverse Primer Sequence | No. |

| | | | | | |
|---|---|---|---|---|---|
| nitrogen | YIR028W | TATTATGAGCTCGAGATACGTTCT | 1 | TATTATACTAGTTCTCGTCTTTGT | 2 |
| limitation | | CCAGCGTATGTATTTCAT | | TGATGTTTTATATCACAAGATGTA | |
| | | | | G | |
| | NSR1 | TATTATCCCGGGGAGATTCCAAAC | 3 | TATTATGGATCCCTTATTTTATCC | 4 |
| | | TGGTTCATTGAAATAGGC | | TGCCTGGGTTGAGTGAT | |
| phosphorus | YAR071W | TATTATGAGCTCGGTGCTGTGACC | 5 | TATTATACTAGTTGGTATTTCTGA | 6 |
| limitation | | GTTTCCAATACG | | TGATGTTCTTGCTCTCTTTG | |
| | RPS22B | TATTATCCCGGGACTGCAACTATT | 7 | TATTATGGATCCTTTTTACCTAAT | 8 |
| | | CTTACAATCTTTCATTTAC | | TACTATGTTTTGAAACGTTAG | |
| sulfur | YFL055W | TATTATGAGCTCTGTTCACGCCCT | 9 | TATTATACTAGTTAGCGAGGATTG | 10 |
| limitation | | CTACGAACCATG | | CTGAAATCTTGTATATTTTCAG | |
| | SSU1 | CCCGGGGCCACGTTCTAAACTAAC | 11 | ATGGATCCTTTTTTCTTGTACTTG | 12 |
| | | TA | | TCTTCTC | |

According to the embodiment of claim 14, the marker gene codes for an enzyme that can be detected by a simple color reaction, for example, β galactosidase, alkaline phosphatase, horseradish peroxidase. The invention encompasses also marker genes that code for enzymes that cause acidification of the medium. The pH shift is converted into a signal by means of a fluorescent protein whose fluorescence depends on the pH value of the surroundings and the signal is detected by a photodetector. Preferably used for this purpose are pHluorins. According to the embodiment of claim 15 the marker gene codes for a luciferase. Luciferases are proteins that are capable of bioluminescence and in the presence of luciferins emit light that can then be detected by the photodetector.

Moreover, the invention comprises marker genes that code for proteases that decompose fluorescent proteins. In this way, based on the signal to be detected the decrease of fluorescence of the whole-cell sensor is measurable. Preferably, proteases are used that, aside from the fluorescent protein, do not attack any other targets in the yeast cell in order to not impair the vitality of the cell. Especially preferred is the use of TEV protease. The corresponding fluorescent proteins must be modified optionally by means of recombinant DNA techniques such that they contain the recognition sequence for the corresponding protease and are therefore decomposable.

According to the embodiment of claim 16, the marker gene codes for a fluorescent protein wherein the expression of the corresponding marker protein upon limitation of bio-available nitrogen, phosphorus and/or sulfur in the medium will vary which leads to an increase or decrease of the fluorescence of the respective yeast cell. Preferred is the use of the genes that code for the fluorescent proteins GFP, YFP, CFP, BFP, RFP, DsRed, PhiYFP, JRed, emGFP (“Emerald Green”), Azami-Green, Zs-Green or AmCyan 1. Preferred is the use of proteins that have been modified such they fluoresce especially strongly. e.g. eGFP, eYFP, TagCFP, TagGFP, TagYFP, TagRFP and TagFP365. Furthermore, such fluorescent proteins are preferred whose amino acids sequence has been modified in that they begin to fluoresce as quickly as possible after their formation. Preferred in this context is TurboGFP, TurboYFP, TurboRFP, TurboFP602, TurboFP635, and dsRed-Express.

According to the embodiment of claim 17, the marker gene codes for a green (for example, GFP), yellow (for example, YFP), blue (for example, BFP), cyan (for example, CFP), or red (for example, dsRed) fluorescent protein. According to the embodiment of claim 18 the marker gene codes for a fluorescent protein with limited half-life. In this way, a fast response time is ensured for a decrease of transcription.

Such a limited half-life can be achieved, for example, by changing the N-terminal amino acid or the introduction of a signal sequence into the amino acid sequence of the protein that is coded by the marker gene so that the stability of the protein is lowered and its half-life is reduced. Preferred is the use of a so-called PEST domain for the destabilization of the protein that is coded by the marker gene that causes a fast decomposition of the protein by the ubiquitin system of the cell. Such PEST domains are known from many proteins. Preferred is the use of the PEST domain of the G1 cyclin Cln2p of Saccharomyces cerevisiae. For this purpose, onto the 3′ end of the coding sequence of the marker gene the coding sequence (SEQ ID NO 13) of the 178 carboxy-terminal amino acids of Cln2p (SEQ ID NO 14) and a stop codon are attached.

```
Cln2p-Pest-Sequenz (SEQ ID NO. 13/14):
GCATCCAACTTGAACATTTCGAGAAAGCTTACCATATGAACCCCATCATGCTCTTTCGAAAATTCAAATAGCACA
  A S H L N I S K K L T I S T P S C S F E N S H S T
TCCATTCCTTCGCCCGCTTCCTCATCTCAAAGCCACACTCCAATGAGAAACATGAGCTCACTCTCTGATAACAGC
  S I P S P A S S S Q S H T P M R N M S S L S D N S
GTTTTCAGCCGGAATATGGAACAATCATCACCAATCACTCCAAGTATGTACCAATTTGGTCAGCAGCAGTCAAAC
  V F S R N M E Q S S P I T P S M Y Q F G Q Q Q S N
AGTATATGTGGTAGCACCGTTAGTGTGAATAGTCTGGTGAATACAAATAACAAACAAAGGATCTACGAACAAATC
  S I C G S T V S V N S L V N T N N K Q R I Y E Q I
ACGGGTCCTAACAGCAATAACGCAACCAATGATTATATTGATTTGCTAAACCTAAATGAGTCTAACAAGGAAAAC
  T G P N S N N A T N D Y I D L L N L N E S N K E N
CAAAATCCCGCAACGGCGCATTACCTCAATGGGGGCCCACCCAAGACAAGCTTCATTAACCATGGAATGTTCCCC
  Q N P A T A H Y L N G G P P K T S F I N H G M F P
TCGCCAACTGGGACCATAAATAGCGGTAAATCTAGCAGTGCCTCATCTTTAATTTCTTTTGGTATGGGCAATACC
  S P T G T I N S G K S S S A S S L I S F G M G N T
CAAGTAATATAG
  Q V I −
```

According to the embodiment of claim 19 the employed yeast cells are cells that have been genetically modified such that their growth can be controlled in a targeted fashion. This enables advantageously culturing the required quantity of yeast cells for producing biosensors under so-called permissive conditions and, after embedding of the yeast cells, the yeast cells are prevented from dividing further by adjusting restrictive conditions. In this way, the pressure exerted within the matrix as a result of the vegetative growth of the cells is advantageously avoided which pressure impairs the durability of the biosensors as well as exerts stress on the immobilized cells and negatively affects their vitality.

Preferred are yeast cells in which the activity of a gene that acts on the cell cycle is controlled in a targeted fashion. Especially preferred are yeast cells in which the activity of the cdc28 gene can be controlled in a targeted fashion. The cdc28 gene is needed by the yeast cell in order to be able to divide. When the gene is not present, the yeast cell can survive but cannot divide further.

The control of gene activity is realized, for example, by the so-called Tet-on system. In this connection, a yeast cell in which the endogenous CDC gene is deleted (a so-called Δcdc28 cell) is transformed by a DNA construct that contains the coding sequence of the CDC28 gene under the control of tet-responsive promoter. At the same time, the construct contains the coding sequence of the reverse tetracycline-controlled transactivator (rtTA) under the control of a constitutive promoter.

Such genetically modified yeast cells express constantly the reverse tetracycline-controlled transactivator. The latter can bind only in the presence of a tetracycline antibiotic such as doxycycline to the tet-responsive promoter and suppress the expression of the gene that is under the control of the tet-responsive promoter. In order to culture the cells, to the culturing medium a tetracycline antibiotic is added and this therefore provides permissive conditions. During or after embedding of the yeast cells into the xerogel the tetracycline antibiotic is washed out and in this way restrictive conditions for the yeast are provided. The reverse tetracycline-controlled transactivator can no longer activate the expression of the CDC28 gene. The yeast cells therefore can no longer divide.

According to the embodiment of claim 20, cell division cycle (cdc) yeast mutants are used that for permissive temperature grow normally and for restrictive temperature stop growth. For example, several temperature sensitive (ts) alleles of the CDC28 gene of Saccharomyces cerevisiae are known. For example, six different ts alleles have been identified that enable normal growth of the yeasts at 23° C. but prevent growth at 37° C. (Lörincz and Reed, 1986). Moreover, temperature-sensitive mutations are known in which the permissive temperature is higher than the restrictive temperature. They are referred to as cold-sensitive (cs) mutations.

Advantageously by utilization of such mutants at permissive temperature first the required biomass is generated while the yeast cells stop growth at restrictive temperature. When such mutants are used for whole-cell sensors, in case of thermosensitive mutants the cells can be cultured advantageously at approximately 25° C. until the desired biomass is reached and then embedded. For restrictive temperature of, for example, 37° C., a temperature that is ideal for fermentation of Escherichia coli, no growth of yeasts will occur anymore even though the cells are physiologically active (Lörincz, A. and Reed, S. I. Sequence analysis of temperature-sensitive mutations in the Saccharomyces cerevisiae gene CDC28. Mol. Cell. Biol. (1986) 6:4099-4103). Yeasts are preferably used that support the temperature-sensitive alleles cdc28-4, cdc28-6, cdc28-9, cdc28-13, cdc28-16, cdc28-17, cdc28-18 and cdc28-19.

For applications in which the yeasts are to be used at low temperatures such as room temperature, cold-sensitive mutants are used that are cultured at high temperatures and after embedding are kept at low temperatures and in this way have no division activity anymore.

According to the embodiment of claim 21 a combination of a green (for example, GFP), a yellow (for example, YFP), a blue (for example, BFP), a cyan (for example, CFP) and/or a red (for example, dsRed) fluorescent marker protein is used. Preferred is the use of combinations of proteins whose excitation and emission spectra are separable clearly from one another by measuring technological means. In particular, the combinations GFP and YFP as well as GFP and DsRed are preferred. The expression of the corresponding marker gene varies for a limitation of bio-available nitrogen, phosphorus and/or sulfur in the medium that leads to an increase or decrease of the fluorescent intensities of the respective yeast cell. In this way, limitations of different components can be detected simultaneously. In this way, multi-functional whole-cell sensors are provided.

The ends of two light-guiding fibers according to the embodiment of claim 22 are simultaneously the substrate for the yeast cells or a substrate with the yeast cells is coupled to the light-guiding fibers. Moreover, to the other end of a first light-guiding fiber a radiation source and to the other end of the second light-guiding fiber the photodetector is coupled so that by means of the light rays in the first light-guiding fiber the yeast cells are excited to fluoresce and the fluorescence light that is induced by the bio-available analyte passes through the second light-guiding fiber and impinges on the photodetector wherein no radiation from the radiation source reaches the substrate.

According to the embodiment of claim 23, an end of a light-guiding fiber is the substrate for the yeast cells or the end of the light-guiding fiber is coupled to a substrate provided with the yeast cells. Moreover, the other end of the light-guiding fiber is coupled by a beam change-over switch either to a radiation source or to the photodetector so that either the excitation radiation of the radiation source passes to the substrate through the beam change-over switch and the light-guiding fiber or the fluorescence light passes through the light-guiding fiber and the change-over switch to the photodetector.

According to claim 24, the whole-cell biosensor according to the invention can be used for the following purposes:
control and/or regulation of the availability of bio-available nitrogen, bio-available phosphorus and/or bio-available sulfur in bioreactors with application possibilities in the entire field of “white biotechnology”;
monitoring and/or controlling systems for purifying drinking water, technical process water and/or wastewater in regard to nitrogen, phosphorus and sulfur loads.

With the aid of the following figures and embodiments, the invention will be explained in more detail. It is shown in:

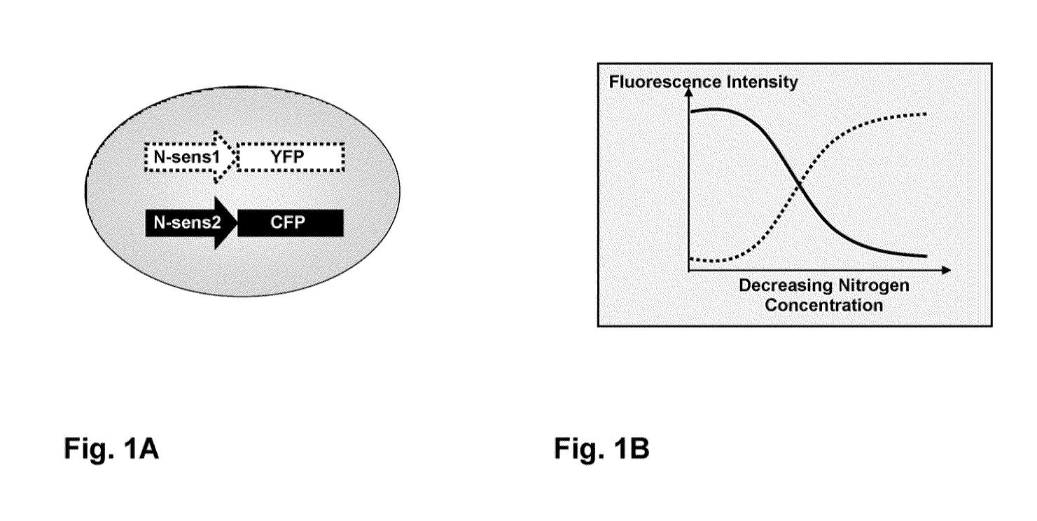

FIG. 1 schematic illustration of a whole-cell sensor

FIG. 1A shows schematically a yeast cell in which the reading frame (YFP) that is coding for YFP is under the control of a promoter (N-sens1) that in case of nitrogen deficiency causes increased transcription. In analogy, the reading frame for CFP is under the control of a promoter (N-sens2) that leads to reduced transcription upon nitrogen deficiency. FIG. 1B shows in a diagram how, based on the respective fluorescence values or their ratios to one another, the actual availability of nitrogen for the cell can be measured.

### EMBODIMENT 1

The genes YAR071W and RPS22B are each specifically transcribed in case of a phosphorus limitation more strongly or weakly, respectively. Cloning of constructs for detection of phosphorus limitation will be described in the following. In analogy, with specific primers (see Table 1) cloning for constructs for detection of sulfur limitation can be realized.

The upstream region of the upregulating gene YAR071W comprising 1,000 base pairs is amplified by means of specific primers (see Table 1) by PCR of genomic DNA for Saccharomyces cerevisiae. Because of the primers the sequence is expanded by a recognition sequence for SacI at the 5′ end and by the recognition sequence for SpeI at the 3′ end. By means of these recognition sequences, a directed insertion into the high copy number vector p426, referred to in the following as p426YAR071W, is provided. For the down-regulating gene RPS22B the regulatory region of the gene is also amplified by PCR with the specific primers listed in Table 1. The amplified region comprises the 1,000 base pairs that are immediately in front of the reading frame of the gene. By means of the employed primers on the RPS22B promoter fragment at the 5′ end and at the 3′ end recognition sequences for the restriction endonucleases SmaI and BamHI are added. The high copy number vector p424GPD is cut by EclI and BamHI so that the obtained GPD promoter is removed. EclI and SmaI produce blunt end fragments that can be ligated with one another. In this way, incorporation of the RPS22B promoter fragment into the plasmid is realized, referred to in the following as p42rRPS22B.

In the second step the reading frame of the marker gene is subjected to the control of the YAR071 W promoter or RPS22B promoter in the plasmids p426YAR071W and p242RPS22B. For this purpose, the sequence of the marker gene is amplified with primers that expand the fragment at the 5′ end by a SpeI or EcoRI and at the 3′ end by a SalI restriction site. The coding sequence for YFP is subjected to the control of the YAR071W promoter and the coding sequence for CFP is subjected to the control of RPS22B promoter. Subsequently, cloning of the corresponding fragments with the aforementioned restriction sites into the vectors p426YAR071W or p424RPS22B takes place. The correct sequence of the cloned fragments is checked and confirmed by means of DNA sequence analysis.

The vector p426 contains the URA3 gene and p424 the TRP1 gene of Saccharomyces cerevisiae for selection of corresponding auxotrophic strains. The produced constructs (p426YAR071W and p424RPS22B) are transformed into the yeast strain W303 (Mat a, ade2-1, his3-1, his3-15, leu2-3, leu2-112, trp1-1, ura3-1) and transformands are selected. In case of phosphorus limitation the transcription and the expression of YFP are greatly increased in such yeast cells while that of CFP is correspondingly reduced.

For fixation of the yeast cells in a porous and optically transparent silicon dioxide xerogel a mixture of yeast cells and a silicon dioxide xerogel is deposited on a light-guiding fiber by means of a known sol-gel process.

Such a coating solution is produced in the following way:
(1) Production of an aqueous SiO₂ nanosol:
100 ml tetraethoxy silane, 400 ml ethanol, and 200 ml 0.01 M hydrochloric acid are stirred at room temperature for 20 hours. 500 ml water are added to this solution and a strong air stream is passed through until a final volume of 700 ml is reached. The SiO₂ sol (pH 3-4) contains approximately 4.2% SiO₂ with an average particle diameter of approximately 6 mm.
(2) Production of the coating solution:
In 100 ml of the aqueous SiO₂ nanosol 2 g of yeast cells (wet weight) are dispersed under stirring or ultrasound for 30 minutes. As a dispersion aid wetting agents (for example, 1 ml 5% aqueous Tween 80 solution) can be used. With this cell suspension the aforementioned glass supports are coated by a dip-coating process (30 cm/min.) and dried in air.

The mechanical stability of these structures enables advantageously coupling of the whole-cell sensor with the reaction space (fermenter) to be examined in a measuring cell in the context of near-line diagnostics. In special embodiments such as a light-guiding fiber the whole-cell sensor can also be directly mounted in the reaction space (fermenter).

The terminal areas of two light-guiding fibers are positioned adjacent to one another. The ends of the light-guiding fibers are simultaneously the substrate for the yeast cells. In one embodiment, also a separate substrate can be coupled to the light-guiding fibers. At the other end of a first light-guiding fiber the radiation source and at the other end of the second light-guiding fiber the photodetector are coupled. By means of the light rays in the first light-guiding fiber the yeast cells are caused to fluoresce. The resulting induced fluorescence light passes through the second light-guiding fiber and impinges on the photodetector; no radiation of the radiation source will reach the substrate. For this purpose, the data processing system is connected to a change-over switch for the radiation source or an aperture for the beam path downstream of the radiation source so that the radiation of the radiation source can reach in a pulsed fashion the substrate. The photodetector is connected to the data processing system so that by means of the fluorescence light detected by the photodetector in combination with the downstream data processing system the contents of bio-available analytes can be determined.

In one embodiment, the end of the light-guiding fiber can be the substrate or the end of the light-guiding fiber can be coupled to the substrate. The other end of the light-guiding fiber is coupled by means of a beam change-over switch either to the radiation source or the photodetector so that either the excitation radiation passes through the beam change-over switch and the light-guiding fiber to impinge on the substrate or the fluorescence light passes through the light-guiding fiber and the beam change-over switch and impinges on the photodetector. A beam change-over switch is for example a folding mirror. The photodetector and the drive for the folding mirror are connected to the data processing system.

For this purpose, the radiation source is advantageously an electromagnetic radiation source for electromagnetic radiation in the form of light in the visible range and in the adjoining wavelength ranges of infrared or ultraviolet.

The substrate that is coupled with the light-guiding fiber/fibers can also be a coated transparent tubular structure to which the light-guiding fiber is coupled

### EMBODIMENT 2

The gene YIR028W in case of nitrogen limitation is transcribed specifically much stronger, the gene NSR1 in case of nitrogen limitation is transcribed specifically much weaker. Based on genomic DNA of Saccharomyces cerevisiae with specific primers the regulatory regions of the genes at the 5′ end including their promoters are PCR amplified (see Table 1). By means of the primers the restriction sites are fused to the fragments that are used for cloning in the vector pUC18. The correct sequence of cloned fragments is verified by means of DNA sequence analysis.

The reading frame of the yellow-fluorescent protein (YFP) and of the cyan-fluorescent protein (CFP) are PCR amplified with DNA of the plasmids pFA6a-EYFP bzw. pFA6a-ECFP (Driesche et al., 2005) as templates. By means of overlap extension PCR (Pogulis et al., 1996) the reading frame of the YFP is subjected to the control of the YIR028W promoter while the reading frame of CFP is subjected to the control of the NSR1 promoter. The resulting fragments are cloned into the single copy vectors pRS415 bzw. pRS416 (Sikorski and Hieter, 1989). The vector pRS415 supports the LEU2 gene and pRS416 supports the URA3 gene of S. cerevisiae for selection in corresponding auxotrophic strains. The resulting constructs (pRS415-YIR028W-YFP and pRS416-NSR1-CFP) are transformed into the yeast strain W303 (Mat a, ade2-1, his3-1, his3-15, leu2-3, leu2-112, trp1-1, ura3-1) and transformands are selected. In case of nitrogen limitation in such yeast cells the transcription and expression of the YFP are greatly increased and that of CFP correspondingly decreased.

Subsequently, the yeast cells are embedded in an envelope structure (duplex embedding) wherein the inner envelope is comprised of alginate and the outer envelope is a porous silicon dioxide xerogel layer. The duplex embedding is realized by means of a sequential coating by utilizing a nanoplotter. In this connection, the yeast cells are embedded before plotting into an aqueous alginate solution (alginate concentration 2 percent by weight; the yeast proportion varies typically between 5 to 25 percent by volume).

By means of the nanoplotter defined volumes of the yeast cells embedded in the alginate are applied dot by dot onto a flat glass support. By using a nanoplotter and by means of selection of the applied volumes as well as a defined concentration of yeast cells, the quantity of the yeast cells applied to the glass support can be selected in a targeted fashion. Subsequently, the nanoplotter applies onto the yeast cells embedded in the alginate a nanosol layer that is subsequently transformed by drying into a xerogel.

The mechanical stability of such structures enables advantageously the introduction of the whole-cell sensor into a measuring cell that is coupled immediately with the reaction space (fermenter) to be examined in the context of near-line diagnostics.

### EMBODIMENT 3

The yeast cells are produced as disclosed in connection with Embodiments 1 and 2.

Subsequently, the yeast cells applied on a flat glass support are introduced into a temperature-controlled and light-microscopically observed measuring cell of a microfluidic system. In this way, a controlled introduction of the medium to be analyzed into the measuring cell by means of the microfluidic systems (off-line diagnostics) is enabled. Advantageously, the temperature adjustment in the fermenter and in measuring cell are independent from one another.

What is claimed is:

1.-14. (canceled)

25. A whole-cell sensor for detecting in a medium bio-available nitrogen, phosphorus, and sulfur, each individually or in at least one combination, the whole-cell sensor comprised of gene-technologically modified yeast cells and a xerogel matrix, wherein the yeast cells are immobilized in a xerogel matrix, wherein the yeast cells contain at least one marker gene under the control of a promoter of a gene whose transcription greatly increases or greatly decreases in case of nitrogen deficiency, phosphorus deficiency or sulfur deficiency, and wherein the yeast cells are coupled at least to one signal detector.

26. The whole-cell sensor according to claim 25, wherein the xerogel is an inorganic xerogel comprised of silicon dioxide, alkylated silicon dioxide, titanium dioxide, aluminum oxide, or mixtures thereof.

27. The whole-cell sensor according to claim 25, wherein the xerogel is an inorganic xerogel that is produced by a sol-gel process.

28. The whole-cell sensor according to claim 25, wherein the xerogel and the yeast cells are applied onto a substrate.

29. The whole-cell sensor according to claim 28, wherein the substrate is at least one light-guiding fiber, a flat glass support, glass beads, or another shaped body of glass selected from hollow spheres, rods, and tubes, or ceramic granules.

30. The whole-cell sensor according to claim 25, further comprising an envelope structure wherein the yeast cells are a component of the envelope structure that encloses at least partially a cavity.

31. The whole-cell sensor according to claim 30, wherein the envelope structure is comprised of a base body with an inner layer of a biological hydrogel and an outer layer of a porous and optically transparent xerogel, wherein the layers are applied at least section-wise.

32. The whole-cell sensor according to 25, wherein the yeast cells are located at least on one surface in a transparent measuring cell and wherein the measuring cell has devices for supplying and removing the medium.

33. The whole-cell sensor according to claim 32, wherein the measuring cell is coupled with a heating device.

34. The whole-cell sensor according to claim 25, wherein the signal detector is a photodetector in the form of a solid state image sensor with photoresistors, photodiodes or phototransistors and wherein the solid state image sensor is connected to a data processing system.

35. The whole-cell sensor according to claim 34, comprising at least one lens that is located in a beam path between the yeast cells and the photodetector.

36. The whole-cell sensor according to claim 25, comprising a radiation source, wherein the yeast cells are coupled with the radiation source such that electromagnetic rays impinge on the yeast cells and the yeast cells fluoresce.

37. The whole-cell sensor according to claim 25, wherein the marker gene is subjected to the control of a promoter that is selected from the promoters of the genes YIR028W, YJR152W, YKR034W, YAR071W, YHR136C, YFL055W, YLL057C, NSR1, FET3, HIP1, YDR508C, RPS22B, YBRO99C, IPT1, SSU1, SOL1 and CTR1 of Saccharomyces cerevisiae.

38. The whole-cell sensor according to claim 25, wherein the marker gene codes for an enzyme that is detectable by a simple color reaction.

39. The whole-cell sensor according to claim 25, wherein the marker gene codes for a luciferase.

40. The whole-cell sensor according to claim 25, wherein the marker gene codes for a fluorescent protein, wherein the expression of the protein that is coded by the marker gene varies in case of limitation of bio-available nitrogen, phosphorus and/or sulfur in the medium, leading to an increase or decrease of the fluorescence of the yeast cells.

41. The whole-cell sensor according to claim 40, wherein the marker gene codes for a green, a yellow, a blue, a cyan, or a red fluorescent protein, wherein the expression of the corresponding marker protein varies in case of limitation of bio-available nitrogen, phosphorus and/or sulfur in the medium, leading to an increase or decrease of the fluorescence of the respective yeast cell.

42. The whole-cell sensor according to claim 40, wherein the marker gene codes for a fluorescent protein with limited half-life.

43. The whole-cell sensor according to claim 40, wherein the yeast cells are cell division cycle (cdc) mutants that under permissive conditions grow normally and stop growth under restrictive conditions.

44. The whole-cell sensor according to claim 40, wherein the yeast cells are temperature-sensitive cell division cycle (cdc) mutants that under permissive temperature grow normally and stop growth under restrictive temperature.

45. The whole-cell sensor according to claim 40, wherein a combination of a green, a yellow, a blue, a cyan, and/or a red fluorescent marker protein is used, wherein the expression of the corresponding marker protein varies in case of limitation of bio-available nitrogen, phosphorus and/or sulfur in the medium, leading to an increase or decrease of fluorescence in the yeast cells so that deficiencies of nitrogen, phosphorus and/or sulfur is detectable simultaneously.

46. The whole-cell sensor according to claim 25, comprising first and second light-guiding fibers wherein first ends of the light-guiding fibers are a substrate for the yeast cells or a substrate with the yeast cells is coupled to the first ends of the light-guiding fibers, wherein to a second end of the first light-guiding fiber a radiation source is coupled and to the second end of the second light-guiding fiber a photodetector is coupled so that light rays emitted by the radiation source excite the yeast cells to fluoresce and the induced fluorescent light that is proportional to the nitrogen proportion passes through the second light-guiding fiber to impinge on the photodetector, wherein no radiation from the radiation source reaches the substrate.

47. The whole-cell sensor according to claim 25, wherein a first end of a light-guiding fiber is a substrate for the yeast cells or the first end of the light-guiding fiber is coupled to a substrate provided with the yeast cells, wherein a second end of the light-guiding fiber is coupled by a beam change-over switch either to a radiation source or a photodetector so that either the radiation of the radiation source for exciting the yeast cells passes through the beam change-over switch and the light-guiding fiber and impinges on the substrate or the fluorescent light of the yeast cells passes through the light-guiding fiber and the beam change-over switch and impinges on the photodetector.

48. The whole-cell sensor according to claim 25 adapted to control or govern the availability of bio-available nitrogen, bio-available phosphorus and/or bio-available sulfur in bioreactors.

49. The whole-cell sensor according to claim 25 adapted to monitor and/or control systems for purifying drinking water, technical process water or waste water with regard to nitrogen, phosphorus, and sulfur loading.
